(19) United States
(12) Patent Application Publication
(10) Pub. No.: US 2004/0044069 A1
(43) Pub. Date: Mar. 4, 2004

(54) Anti-virus compounds

(76) Inventors: Tsu-An Hsu, Taipei (TW); Hsing-Pang Hsieh, Taipei (TW); Li-Jung Juan, Taipei (TW); Sui-Yuan Chang, Taipei (TW); Yueh-Hsiung Kuo, Taipei (TW)

Correspondence Address: FISH & RICHARDSON PC, 225 FRANKLIN ST, BOSTON, MA, 02110, US

(21) Appl. No.: 10/445,268

(22) Filed: May 23, 2003

Related U.S. Application Data
(60) Provisional application No. 60/382,692, filed on May 23, 2002.

(51) Int. Cl. A61K 31/335; A61K 31/235; A61K 31/24

(52) U.S. Cl. 514/463; 514/532; 514/534

(57) ABSTRACT

This invention features a method for treating infection by herpesvirus. The method includes administering to a subject in need thereof an effective amount of an arylnaphthalene compound of formula (I):

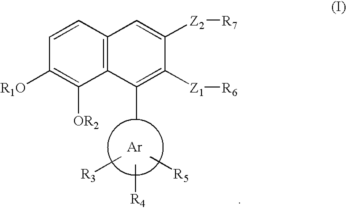

Each of R₁ and R₂, independently is R or C(O)R; or R₁ and R₂ taken together is (CH₂)ₘ; each of R₃, R₄, and R₅, independently, is R, OR, C(O)R, or OC(O)R; or any two of R₃, R₄, and R₅ taken together is O(CH₂)ₙO; Ar is aryl; each of Z₁ and Z₂, independently, is CH₂ or C(O); and each of R₆ and R₇, independently, is R, OR, SR, or NRR′; or R₆ and R₇ taken together is O, S, or NR; in which each of R and R′, independently, is H, alkyl, (CH₂)ₒ-aryl, (CH₂)ₚ-heteroaryl, cyclyl, or heterocyclyl; each of m and n, independently, is 1, 2, 3, or 4; and each of o and p, independently, is 0, 1, 2, 3, 4, 5, or 6.

### CROSS REFERENCE TO RELATED APPLICATIONS

Pursuant to 35 USC § 119(e), this application claims the benefit of prior U.S. provisional application No. 60/382,692, filed May 23, 2002.

### BACKGROUND

Herpesviruses infect all known vertebrate species, and are believed to be extremely ancient. The name “herpes” comes from the Greek “herpein” (“to creep”), referring to chronic, latent, and recurrent infections. To date, there are 8 known human herpesviruses. They are divided into 3 sub-families: α-herpesvirinae [herpesvirus 1 (i.e., simplexvirus 1), herpesvirus 2 (i.e., simplexvirus 2), and herpesvirus 3 (i.e., varicellovirus)], β-herpesvirinae [herpesvirus 5 (i.e., cytomegalovirus), herpesvirus 6, and herpesvirus 7], and γ-herpesvirinae [herpesvirus 4 (i.e., lymphocryptovirus) and herpesvirus 8]. See, e.g., Knipe & Howley et al. (2001) Fields Virology, Ch. 71, Lippincott Williams & Wilkins 2: 2493-2523.

Millions of people are infected with herpesviruses worldwide. For example, 60-98% of adult individuals are latently infected with human cytomegalovirus (“HCMV”). Britt & Alford (1996) Fields Virology, Fields et al. Philadelphia, Lippincott-Raven Publishers 2: 2493-2523. Primary infection generally occurs in a subclinical fashion in early childhood, with subsequent lifelong persistence of infection. Gold & Nankervis (1982) Sci. Am. 285(5): 56-63. HCMV is a major cause of congenital or neonatal infections, which can be severe (fatal in 5-10% of cases), in part because of its ability to cross the placenta. It is reactivated in much the same way as the other human herpesviruses, and can cause pneumonia, as well as retinitis, in immune-compromised individuals. See, e.g., Fowler et al. (1992) N Engl J Med 326(10): 663-7; Boppana et al. (1992) Pediatr Infect Dis J 11(2): 93-9; Boppana et al. (1997) Pediatrics 99(3): 409-14; and Torriani et al. (2000) Aids 14(2): 173-80. For those immune-compromised individuals, such as transplant recipients and AIDS patients, HCMV is known as the major cause of morbidity and mortality. See, e.g., Dummer (1990) Rev Infect Dis 12 Suppl 7: S767-75; Enright et al. (1993) Transplantation 55(6): 1339-46; Davis et al. (1987) Proc Natl Acad Sci USA 84(23): 8642-6; and Webster (1991) J Acquir Immune Defic Syndr 4 Suppl 1: S47-52.

Antiviral drugs include guanosine analogs (e.g., acyclovir, ganciclovir, or famciclovir) and nucleotide analogs (e.g., cidofovir). Herpesviruses have been found to become resistant to these drugs. Thus, there is a need to identify compounds that can efficiently treat infection by herpesviruses.

### SUMMARY

The present invention relates to use of arylnaphthalene compounds as an antiviral compound to treat herpesviral infection.

In one aspect, this invention features a method for treating infection by a β-herpesvirinae (i.e., herpesvirus 5, 6, or 7). The method includes administering to a subject in need thereof an effective amount of an arylnaphthalene compound of formula (I):

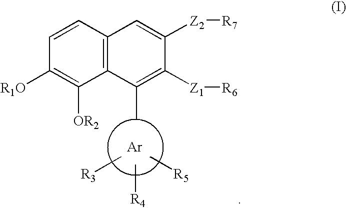

Each of R₁ and R₂, independently is R or C(O)R; or R₁ and R₂ taken together is (CH₂)ₘ; each of R₃, R₄, and R₅, independently, is R, OR, C(O)R, or OC(O)R; or any two of R₃, R₄, and R₅ taken together is O(CH₂)ₙO; Ar is aryl; each of Z₁ and Z₂, independently, is CH₂ or C(O); and each of R₆ and R₇, independently, is R, OR, SR, or NRR′; or R₆ and R₇ taken together is O, S, or NR; in which each of R and R′, independently, is H, alkyl, (CH₂)ₒ-aryl, (CH₂)ₚ-heteroaryl, cyclyl, or heterocyclyl; each of m and n, independently, is 1, 2, 3, or 4; and each of o and p, independently, is 0, 1, 2, 3, 4, 5, or 6. Note that the left atom shown in any substituted group described above is closest to the naphthyl ring. Each of (CH₂)ₘ, (CH₂)ₙ, (CH₂)ₒ, and (CH₂)ₚ, independently, is either linear or branched. Also note that when there are more than one R-containing substituted groups in an arylnaphthalene compound, the R moieties can be the same or different. The same rule applies to other similar situations.

A subset of the just-described compounds are those in which Ar is phenyl substituted with R₃, R₄, and R₅ at positions 3, 4, and 5, respectively. In these compounds, R₃ and R₄ taken together can be OCH₂O, R₅ can be H, Z₁ can be CH₂, Z₂ can be C(O), and R₆ and R₇ taken together can be 0 or R₆ can be OH and R₇ can be NHCH₃.

Another subset of the compounds are those in which Z₁ is CH₂ and Z₂ is C(O). In these compounds, R₆ and R₇ taken together can be O or R₆ can be OH and R₇ can be NHCH₃, Ar can be phenyl substituted with R₃, R₄, and R₅ at positions 3, 4, and 5, respectively; R₁ and R₂ taken together can be CH₂; and R₃ and R₄ taken together can be OCH₂O.

A further subset of the compounds are those in which R₁ and R₂ taken together is CH₂. In these compounds, Ar can be phenyl substituted with R₃, R₄, and R₅ at positions 3, 4, and 5, respectively; R₃ and R₄ taken together can be OCH₂O; R₅ can be H; R₆ and R₇ taken together can be O or R₆ can be OH and R₇ can be NHCH₃; Z₁ can be CH₂; and Z₂ can be C(O).

Shown below are the seven exemplary compounds, i.e., Compounds 1-7:

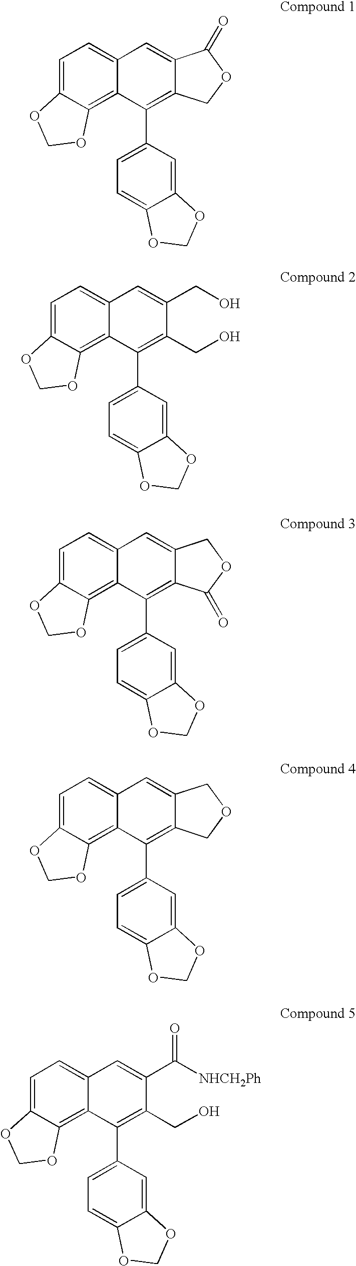

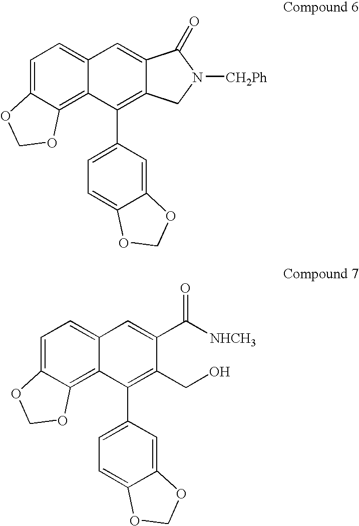

Alkyl, aryl, heteroaryl, cyclyl, heterocyclyl, and the naphthyl ring mentioned herein include both substituted and unsubstituted moieties. The term “substituted” refers to one or more substituents (which may be the same or different), each replacing a hydrogen atom. The substituted moieties may be the same as or different from R₁, R₂, R₃, R₄, R₅, R₆, or R₇. Examples of substituents include, but are not limited to, halogen, hydroxyl, amino, alkylamino, arylamino, dialkylamino, diarylamino, cyano, nitro, mercapto, carbonyl, carbamido, carbamyl, carboxyl, thioureido, thiocyanato, sulfoamido, C₁˜C₆ alkyl, C₁˜C₆ alkenyl, C₁˜C₆ alkoxy, aryl, heteroaryl, cyclyl, heterocyclyl, wherein alkyl, alkenyl, alkoxy, aryl, heteroaryl cyclyl, and heterocyclyl are optionally substituted with C₁˜C₆ alkyl, aryl, heteroaryl, halogen, hydroxyl, amino, mercapto, cyano, or nitro.

The term “aryl” refers to a hydrocarbon ring system having at least one aromatic ring. Examples of aryl moieties include, but are not limited to, phenyl, naphthyl, and pyrenyl. The term “heteroaryl” refers to a hydrocarbon ring system having at least one aromatic ring which contains at least one heteroatom such as O, N, or S. Examples of heteroaryl moieties include, but are not limited to, pyridinyl, carbazolyl, and indolyl. The terms “cyclyl” and “heterocyclyl” refer to partially and fully saturated mono-, bi-, or tri-cyclic rings having from 4 to 14 ring atoms. A heterocyclyl ring contains one or more heteroatoms. Exemplary cyclyl and heterocyclyl rings are cycylohexane, piperidine, piperazine, morpholine, thiomorpholine, and 1,4-oxazepane.

The arylnaphthalene compounds described above include the compounds themselves, as well as their salts and their prodrugs, if applicable. Such salts, for example, can be formed by interaction between a negatively charged substituent (e.g., carboxylate) on an arylnaphthalene compound and a cation. Suitable cations include, but are not limited to, sodium ion, potassium ion, magnesium ion, calcium ion, and an ammonium cation (e.g., teteramethylammonium ion). Likewise, a positively charged substituent (e.g., amino) can form a salt with a negatively charged counterion. Suitable counterions include, but are not limited to, chloride, bromide, iodide, sulfate, nitrate, phosphate, or acetate. Examples of prodrugs include esters and other pharmaceutically acceptable derivatives, which, upon administration to a subject, are capable of providing arylnaphthalene compounds described above.

The method described above can further include concurrently administering to the subject with an effective amount of another antiviral compound (e.g., acyclovir, gancicolovir, famciclovir, cidofovir, foscarnet sodium, penciclovir, valaciclovir, vidarabine, or fomivirsen) either by itself or as a component of an antiviral composition.

Acyclovir, acycloguanosine, is sold as ZOVIRAX by GlaxoSmithKline. Gancicolovir, 2-amino-1,9-dihydro-9-[(2-hydroxy-1-(hydroxymethyl)ethoxy)methyl]-6H-purin-6-one, is sold as CYTOVENE by F. Hoffmann La-Roche Ltd. Famciclovir, 2-[2-(2-amino-9H-purin-9-yl)ethyl]-1,3-propanediol diacetate (ester), is sold as FAMVIR by GlaxoSmithKline. Cidofovir, (S)-[[2-(4-amino-2-oxo-1(2H)-pyrimidinyl)-1-(hydroxymethyl)ethoxy]methyl]phosphonic acid, is sold as VISTIDE by Gilead Science. Foscarnet sodium, dihyoxyphospinecarboxylic acid oxide trisodium salt, is sold as FOSCAVIR by AstraZeneca. Penciclovir, 2-amino-1,9-dihydro-9-[4-hydroxy-3-(hydroxymethyl)butyl]-6H-purin-6-one, is sold as DENAVIR by GlaxoSmithKline. Valaciclovir, L-valine 2-[(2-amino-1,6-dihydro-6-oxo-9H-purin-9-yl)methoxy]ethyl ester (as hydrochloride salt), is sold as VALTREX by GlaxoSmithKline. Vidarabine, 9-β-D-arabinofuranosyl-9H-6-amine, is sold as VIRA-A by Pfizer. Fomivirsen, a phosphorothioate oligonucleotide having the sequence: 5′-GCG TTT GCT CTT CTT CTT GCG-3′, is sold as VITRAVENE by Isis Pharmaceuticals. All of the just-described compounds can be in the form of a pharmaceutically acceptable salt, if applicable.

As used herein, “concurrent administration” refers to administering an arylnaphthalene compound and a second antiviral compound as a mixture, or administering each by itself either at the same time or at different times. Typically, the second antiviral compound is administered via an established route in an established amount. The arylnaphthalene compound, on the other hand, can be administered orally, parenterally, by inhalation spray, or via an implanted reservoir, in an amount of about 0.01 mg/Kg to about 1000 mg/Kg. Effective doses also vary, as recognized by those skilled in the art, depending on route of administration, excipient usage, and the possibility of co-usage with any other therapeutic agent.

In another aspect, this invention features a method for treating infection by a γ-herpesvirinae (i.e., herpesvirus 4 or 8) or herpesvirus 3 with one or more of the above-described arylnaphthalene compounds.

This invention also features a method for treating infection by an α or γ-herpesvirinae (i.e., herpesvirus 1, 2, 3, 4, or 8) with both an effective amount of the above-described arylnaphthalene compound and an effective amount of another antiviral compound.

As used herein, the term “treating infection” refers to use of one or more arylnaphthalene compounds for preventing or treating infection by α-, β-, or γ-herpesvirinae, as well as for preventing or treating other diseases or disorders secondary to the herpesviral infection. Those secondary diseases or disorders include, but are not limited to, human cytomegalovirus retinitis, human cytomegalovirus polyneuritis, and human cytomegalovirus carditis.

Also within the scope of this invention is the use of the above-described compounds for the manufacture of a medicament for treating herpesviral infection.

Other features, objects, and advantages of the invention will be apparent from the description and from the claims.

### DETAILED DESCRIPTION

An arylnaphthalene compound used to practice the method of this invention can be prepared by procedures well known to a skilled person in the art (see, e.g., U.S. Pat. No. 6,030,967).

One example is shown in Scheme 1 below:

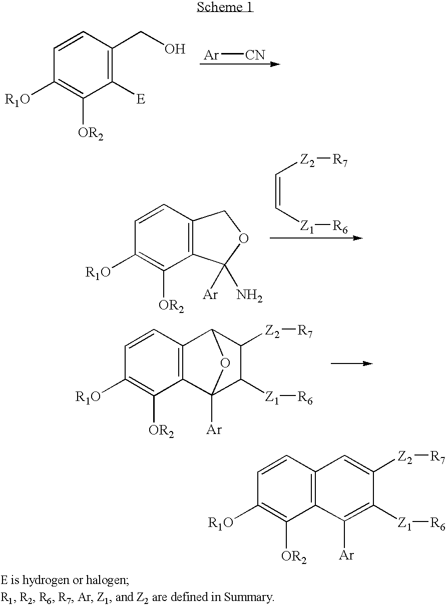

More specifically, an arylnaphthalene compound is prepared by adding an aryl nitrile to a solution containing a substituted phenyl methanol in the presence of a metal catalyst. Subsequently, Z₁-R₆- and Z₂-R₇-substituted ethene is added to the solution, followed by dehydration and aromatization reactions to produce a desired arylnaphthalene compound. See, e.g., Smith et al. (1988) J. Organic Chem. 53: 2942-2953.

Scheme 2 below depicts another route of preparing an arylnaphthalene compound.

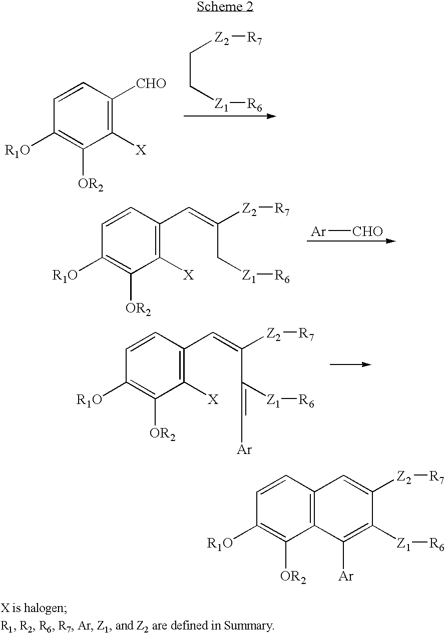

More specifically, an arylnaphthalene compound is prepared by adding Z₁-R₆- and Z₂-R₇-substituted ethane to a substituted phenyl aldehyde, followed by addition of an aryl aldehyde. Then, a palladium-catalyzed benzannulation reaction gives a desired arylnaphthalene compound. See, e.g., Mizufune et al. (2001) Tetrahedron Lett. 42: 437.

The chemicals used in the above-described synthetic routes may include, for example, solvents, reagents, catalysts, and protecting group and deprotecting group reagents. The methods described above may also additionally include steps, either before or after the steps described specifically herein, to add or remove suitable protecting groups in order to ultimately allow synthesis of the arylnaphthalene compound. In addition, various synthetic steps may be performed in an alternate sequence or order to give the desired compounds. Synthetic chemistry transformations and protecting group methodologies (protection and deprotection) useful in synthesizing applicable arylnaphthalene compounds are known in the art and include, for example, those described in R. Larock, Comprehensive Organic Transformations, VCH Publishers (1989); T. W. Greene and P. G. M. Wuts, Protective Groups in Organic Synthesis, 2^{nd} Ed., John Wiley and Sons (1991); L. Fieser and M. Fieser, Fieser and Fieser 's Reagents for Organic Synthesis, John Wiley and Sons (1994); and L. Paquette, ed., Encyclopedia of Reagents for Organic Synthesis, John Wiley and Sons (1995) and subsequent editions thereof.

An arylnaphthalene compound thus synthesized can be further purified by a method such as column chromatography, high pressure liquid chromatography (HPLC), or recrystallization.

In addition to the synthetic procedures described above, some arylnaphthalene compounds, e.g., Compound 1, can be isolated from a natural source by procedures well known to a skilled person in the art (see, e.g., U.S. Pat. No. 6,306,899 B1). For example, an arylnaphthalene compound can be isolated from Taiwania (Taiwania cyptomerioides Hayata) by the following procedures: Taiwania roots are pulverized and extracted with an alcohol, e.g., methanol. The extract is condensed and subsequently extracted with an organic solvent (e.g., chloroform, n-hexane, or ethyl acetate), followed by flash column chromatography and semi-preparative HPLC.

This invention features a method for treating infection by herpesvirus. The method includes administering to a subject in need thereof an effective amount of one or more arylnaphthalene compounds and a pharmaceutically acceptable carrier. The “herpesvirus” includes human herpesviruses and non-human herpesviruses, such as muromegalovirus (i.e., a β-herpesvirinae). The term “treating” is defined as the application or administration of a composition including an arylnaphthalene compound to a subject, who has an infection, a symptom of an infection, a disease or disorder secondary to an infection, or a predisposition toward an infection, with the purpose to cure, alleviate, relieve, remedy, or ameliorate the infection, the symptom of the infection, the disease or disorder secondary to the infection, or the predisposition toward the infection. “An effective amount” is defined as the amount of an arylnaphthalene compound (alone or in combination with another antiviral compound) which, upon administration to a subject in need thereof, is required to confer therapeutic effect on the subject.

To practice the method of the present invention, an arylnaphthalene compound can be administered orally, parenterally (including subcutaneous, intracutaneous, intravenous, intramuscular, intraarticular, intraarterial, intrasynovial, intrasternal, intrathecal, intralesional and intracranial injection, and infusion techniques), by inhalation spray, or via an implanted reservoir.

A composition for oral administration can be any orally acceptable dosage form including, but not limited to, tablets, capsules, emulsions and aqueous suspensions, dispersions and solutions. Commonly used carriers for tablets include lactose and corn starch. Lubricating agents, such as magnesium stearate, are also typically added to tablets. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A sterile injectable composition (e.g., aqueous or oleaginous suspension) can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or di-glycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents.

An inhalation composition can be prepared according to techniques well known in the art of pharmaceutical formulation and can be prepared as solutions in saline, employing benzyl alcohol or other suitable preservatives, absorption promoters to enhance bioavailability, fluorocarbons, and/or other solubilizing or dispersing agents known in the art.

A carrier in a pharmaceutical composition must be “acceptable” in the sense of being compatible with the active ingredient of the formulation (and preferably, capable of stabilizing it) and not deleterious to the subject to be treated. For example, solubilizing agents, such as cyclodextrins (which form specific, more soluble complexes with arylnaphthalene compounds), can be utilized as pharmaceutical excipients for delivery of arylnaphthalene compounds. Examples of other carriers include colloidal silicon dioxide, magnesium stearate, cellulose, sodium lauryl sulfate, and D&C Yellow # 10.

A suitable in vitro assay can be used to preliminarily evaluate the efficacy of an arylnaphthalene compound in inhibiting herpesvirus replication. See examples 8-9 below. In vivo assays can also be performed by following procedures well known in the art to screen for efficacious arylnaphthalene compounds.

Without further elaboration, it is believed that the above description has adequately enabled the present invention. The following specific embodiment is, therefore, to be construed as merely illustrative, and not limitative of the remainder of the disclosure in any way whatsoever. All of the publications, including patents, cited herein are hereby incorporated by reference in their entirety.

### EXAMPLE 1

### Preparation of Compound 1: 10-benzo[1,3]dioxol-5-yl-9H-furo[3′,4′:6,7]naphtho[1,2-d][1,3]dioxol-7-one

Compound 1 was obtained from a natural source (See Wang et al., Quarterly Journal of the Experimental Forestry on National Taiwan University (1997) 11:67-81; and Wang, et al., Quarterly Journal of Chinese Forestry (1998) 31:187-193 for detailed procedures.)

¹H NMR (CDCl₃): δ8.41 (s, 1H); 7.69 (d, J=8.8 Hz, 1H); 7.30 (d, J=8.8 Hz, 1H); 6.87 (d, J=7.2 Hz, 1H); 6.77-6.79 (m, 2H); 6.06 (dd, J=12.0, 1.6 Hz, 2H); 5.94 (dd, J=8.8, 1.2 Hz, 2H); and 5.19 (dd, J=30.8, 15.6 Hz, 2H).

### EXAMPLE 2

### Preparation of Compound 2: (9-benzo[1,3]dioxol-5-yl-7-hydroxymethyl-naphtho[1,2-d][1,3]dioxol-8-yl)-methanol

A solution of compound 1 (300 mg) in dry THF (5 mL) was slowly added to a stirred suspension of LAH (66 mg) in dry THF (3 mL) at 0° C. under nitrogen atmosphere. The reaction mixture was slowly brought to the room temperature and stirred for 6 hours. It was then consecutively treated with 1:1H₂O-THF (5 mL) and 15% aq. NaOH (5 mL). The reaction mixture was then filtered. The filtrate was treated with 10% HCl and then extracted with CH₂Cl₂ (3×50 mL). The combined organic layer was washed with 10% NaHCO₃ solution, dried over MgSO₄, and concentrated under vacuum. Compound 2 was obtained as a colorless solid in 80% yield, which was used as such without further purification. ¹H NMR (CDCl₃): δ7.76 (s, 1H); 7.40 (d, J=8.8 Hz, 1H); 7.16 (d, J=8.8 Hz, 1H); 6.83 (d, J=7.2 Hz, 1H); 6.76 (s, 1H), 6.72 (d, J=7.6 Hz, 1H); 6.02 (d, J=16 Hz, 2H); 5.78 (d, J=4.4 Hz, 2H); 4.88 (s, 2H); 4.59 (d, J=3.6 Hz, 2H); 2.15 (bs, 1H); and 2.11 (bs, 1H).

### EXAMPLE 3

### Preparation of Compound 3: 10-benzo[1,3]dioxol-5-yl-7H-furo[3′,4′:6,7]naphtho[1,2-d][1,3]dioxol-9-one

Silver carbonate-celite (2.0 g) was added to a solution of compound 2 (107 mg) in benzene (100 mL). Solvent was distilled off until the vapor temperature reaches 80° C. The reaction mixture was then heated under reflux for six hours, cooled down to room temperature, and filtered. A yellow-green solid was obtained after removing the solvent. Integration of the ¹H NMR spectrum of the crude oxidation mixture of lactones indicated an approximate ca. 7:3 of compound 1 and compound 3. The mixture was subjected to column chromatography (silica gel) using 33% ethyl acetate in hexane as an eluent to give pure compound 1 and compound 3.

¹H NMR (CDCl₃): δ7.79 (s, 1H); 7.51 (d, J=8.8 Hz, 1H); 7.35 (d, J=8.8 Hz, 1H); 6.86 (d, J=8.0 Hz, 1H); 6.81-6.79 (m, 2H); 6.03 (dd, J=12.0, 1.6 Hz, 2H); 5.89 (dd, J=8.8, 1.2 Hz, 2H); and 5.36 (s, 2H).

### EXAMPLE 4

### Preparation of Compound 4: 10-benzo[1,3]dioxol-5-yl-7,9-dihydro-furo[3′,4′:6,7]naphtho[1,2-d][1,3]dioxole

Compound 2 (100 mg) and p-TSA (6 mg) were refluxed in benzene for 10 h with azeotropic removal of water in a Dean-Stark apparatus. The reaction mixture was cooled and treated with 15% aq. NaOH (20 mL). The organic layer was dried over MgSO₄ and concentrated. The residue (65 mg) thus obtained was purified by column chromatography (silica gel) using ethyl acetate:hexane (1:4) as an eluent to give compound 4 in 60% yield.

¹H NMR (CDCl₃): δ7.59 (s, 1H); 7.33 (d, J=8.4 Hz, 1H); 7.11 (d, J=8.8 Hz, 1H); 6.83 (d, J=8.0 Hz, 1H); 6.74 (s, 1H); 6.72 (d, J=7.6 Hz, 1H); 6.0 (s, 2H); 5.75 (d, J=4.4 Hz, 2H); 4.81 (s, 2H); and 4.63 (s, 2H).

### EXAMPLE 5

### Preparation of Compound 5: 9-benzo[1,3]dioxol-5-yl-8-hydroxymethyl-naphtho[1,2-d][1,3]dioxole-7-carboxylic Acid Benzylamide

Compound 1 (88 mg) was dissolved in dry DMF (5 mL) and followed by addition of redistilled benzyl amine (1.2 mL). The reaction mixture was stirred for 2 days at room temperature under nitrogen atmosphere. The solvent was then removed under vacuum and the residue was subjected to column chromatography (silica gel) using ethyl acetate:hexane (40:60) as an eluent to give compound 5 as a white crystalline solid (46 mg, 52%).

¹H NMR (CDCl₃): δ7.94 (s, 1H); 7.42 (d, J=8.4 Hz, 1H); 7.38-7.29 (m, 5H); 7.19 (d, J=8.0, 1H); 6.83 (d, J=8.0 Hz, 1H); 6.77-6.73 (m, 2H); 6.01 (d, J=22.2 Hz, 2H); 5.81 (d, J=6.4 Hz, 2H); 4.68 (d, J=6.0 Hz, 2H); 4.41 (dd, J=5.2, 1.2 Hz, 2H).

### EXAMPLE 6

### Preparation of Compound 6: 10-benzo[1,3]dioxol-5-yl-8-benzyl-8,9-dihydro-1,3-dioxa-8-aza-dicyclopenta[a,g]naphthalen-7-one

Compound 1 (80 mg) was dissolved in dry methanol (5 mL) and followed by addition of methylamine in methanol (40%) (1.2 mL). The reaction mixture was stirred for 24 hours at room temperature under nitrogen atmosphere. The solvent was then removed under vacuum and the residue was subjected to column chromatography (silica gel) using ethyl acetate:hexane (40:60) as an eluent to give compound 6 as a white crystalline solid.

¹H NMR (CDCl₃): δ8.58 (d, J=8.8 Hz, 1H); 7.99 (s, 1H); 7.61 (d, J=8 Hz, 1H); 7.36 (d, J=8.8 Hz, 1H); 6.92 (d, J=7.2 Hz, 1H); 6.79 (s, 1H); 6.68 (d, J=7.2 Hz, 1H); 6.07 (d, J=9.6 Hz, 2H); 5.84 (d, J=9.6 Hz, 2H); 4.94 (bs, 1H); 4.24 (bs, 2H); and 2.80 (d, J=4.4 Hz, 3H).

### EXAMPLE 7

### Preparation of Compound 7: 9-benzo[1,3]dioxol-5-yl-8-hydroxymethyl-naphtho[1,2-d][1,3]dioxole-7-carboxylic Acid Methylamide

Compound 5 (8 mg) was treated with 70% perchloric acid (3 mL) for 4 hours in an ice bath with occasional stirring. The resultant mixture was poured on 20 grams of ice and 10 mL of cold water. The phthalimidine perchlorate salt thus obtained was collected by filtration and washed with water. The salt was stirred in 5 ml of 6N sodium hydroxide solution for 1 hour. Phthalimidine was extracted with ethyl acetate and dried over MgSO₄. The residue was subjected to column chromatography (silica gel) using ethyl acetate and hexane (40:60) as an eluent to give colorless compound 7 in 51% yield.

¹H NMR (CDCl₃): δ8.50 (s, 1H); 7.83 (s, 1H); 7.51 (d, J=8.4 Hz, 1H); 7.40-7.19 (m, 6H); 7.04 (s, 2H); 6.25 (s, 2H); 6.01 (s, 2H); 4.70 (d, J=6.0 Hz, 2H); and 4.19 (s, 2H).

### EXAMPLE 8

### Inhibition of Human Herpesvirus 5 Replication

Compound 1 was isolated from a unique Taiwanese plant (Taiwania cryptomerioides). See, e.g., Chang et al. (2000) Phytochemistry 55: 227. Antiviral drug ganciclovir (i.e., 9-(2-hydroxyethoxymethyl)guanine, GCV) was purchased from Sigma. Both compounds were solubilized in 100% dimethyl sulfoxide (DMSO) at a concentration of 10 mM and stored at −20° C. The compounds were added to cultures such that the resulting concentrations of DMSO never exceed 0.05%, by volume. To reserve the stability of both compounds, Compound 1 and GCV were never stored in cell culture medium and made fresh for each experiment.

Human cell lines, HEL299 (ATCC CCL-137), MRC-5 (ATCC CCL-171), H1299 (CRL-5803), and ARPE19 (CRL-2302) were grown in respective culture media as indicated by the ATCC. All cell lines were screened periodically for mycoplasma contamination. Two HCMV lab strains, AD169 (VR-538) and the Town strain RC256 (the ATCC VR-2356) were purchased from ATCC. All stocks of HCMV were prepared as described in Spaete & Mocarski (1985) J Virol 54(3): 817-24.

The activities of Compound 1 and GCV against herpesvirus 5 were determined by a plaque reduction assay. All herpesvirus 5 plaque reduction assays were performed with monolayer cultures of MRC-5 cells in 6-well cluster dishes (Costar, Cambridge, Mass.). Briefly, one day before the experiment, MRC-5 cells were seeded into 6-well plates at a final concentration of 5×10⁶ cells/well. On the day of virus inoculation, cells were treated with each test compound at a desired concentration for one hour before virus inoculation. The serially-diluted virus inoculums containing 10⁵ to 10⁰ PFU of herpesvirus 5 per cell were then added into cell cultures and incubated for additional 2 hours at 37° C. After the incubation, the cells were extensively washed with phosphate buffered saline (PBS) three times and then overlayed with the overlay medium containing 0.3% agar in the absence or presence of the test compounds. The cell cultures were maintained in the 37° C. incubator for two weeks. After removing the overlay agar, the cells were stained with crystal violet.

The results showed that herpesvirus 5 growth in the presence of Compound 1 was inhibited. The IC₅₀ value (i.e., 50% infectious concentration) of Compound 1 was below 0.1 μM, which was ten-fold more potent than that of GCV (1 μM). The cytotoxic effect was also determined using the MTS (3-(4,5-dimethylthiazol-2-yl)-5-(3-carboxymethoxyphenyl)-2-(4-sulfophenyl)-2H-tetrazolium, inner salt) assay method described in Goodwin et al. (1995, J. Immunol. Methods. 179: 95). The LC₅₀ value (i.e., half lethal concentration) of Compound 1 was >16 μM. Thus, the SI (i.e., selectivity index=LC₅₀/IC₅₀) of Compound 1 is >160.

### EXAMPLE 9

### Inhibition of Expression of Herpesvirus 5 Immediate Early Protein 2 (IE2 Protein)

Cell-free virus stock, whose titer had been determined by a plaque reduction assay, was used. One day before the experiment, herpesvirus 5-permissive cells were seeded into 6-well plates at a final concentration of 5×10⁶ cells/well. On the day of viral infection, the cells were incubated with or without a test compound (Compound 1 or GCV) for one hour. Subsequently, the cells were washed with PBS twice and serum-free medium were added during virus inoculation. The virus inocula were approximately at a multiplicity of infection (MOI) of 0.1 to 1 PFU/cell depending on which cell line was used. After two hours of incubation with viruses, cells were washed with PBS to remove unbound viruses. The cells were maintained at fresh media with or without test compounds at 37° C. with 5% CO₂ incubator. The cell lysate samples were collected on day 0, 1, 3, 5, and 7 after virus infection.

More specifically, HEL299 cells were infected with herpesvirus 5 lab strain AD169 at an MOI of 1.0. Cell lysates were then collected after 2 hours of virus absorption on day 0, 1, 3, 5, and 7 post-infection. IE2 protein in virally infected cells was expressed rapidly on day 1 and increased dramatically on day 5. The expression of herpesvirus 5 late proteins was also detected on days 5 and 7 post-infection. However, none of these viral proteins was detected in herpesvirus 5-infected cells treated with Compound 1. β-Actin was also expressed as an internal control for determining amounts of proteins in each sample.

To determine whether the absence of IE2 protein expression was due to Compound 1-induced abrogation at the transcriptional level, a reverse transcription (RT) assay was performed to measure the IE2 mRNA in the absence or presence of Compound 1. Basically, HEL299 cells were infected with herpesvirus 5 lab strain AD169 at a MOI of 1.0. Total RNAs were then extracted, collected after 2 hours of virus absorption on day 0, 1, 3, 5, and 7 post-infection. The expression of immediate early (ie) and late (pp150) gene products were determined by amplifying segments spanning at a common region shared by ie genes 1 and 2, and the 5′ end of pp150. As an internal control, a segment spanning β-actin was also amplified. The results showed that, in the absence of Compound 1, the expression of ie transcripts in infected cell lysates was immediately detected on day 1 post-infection. The expression of pp150 transcripts was also detected after day 3. However, in the presence of Compound 1, none of these viral RNA was detected. These results demonstrated that Compound 1 inhibits de novo viral RNA synthesis, indicating that Compound 1 exerts its effects at the very early stages of herpesvirus 5 life cycle.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method for treating infection by a β-herpesvirinae, comprising administering to a subject in need thereof an effective amount of an antiviral compound of formula (I): 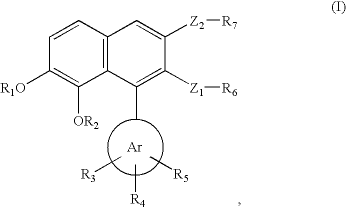
wherein
each of R₁ and R₂, independently is R or C(O)R; or R₁ and R₂ taken together is (CH₂)ₘ;
each of R₃, R₄, and R₅, independently, is R, OR, C(O)R, or OC(O)R; or any two of R₃, R₄, and R₅ taken together is O(CH₂)ₙO;
Ar is aryl;
each of Z₁ and Z₂, independently, is CH₂ or C(O); and
each of R₆ and R₇, independently, is R, OR, SR, or NRR′; or R₆ and R₇ taken together is O, S, or NR;
in which each of R and R′, independently, is H, alkyl, (CH₂)ₒ-aryl, (CH₂)ₚ-heteroaryl, cyclyl, or heterocyclyl; each of m and n, independently, is 1, 2, 3, or 4; and each of o and p, independently, is 0, 1, 2, 3, 4, 5, or 6.

2. The method of claim 1, wherein Ar is phenyl, and is substituted with R₃, R₄, and R₅ at positions 3, 4, and 5, respectively.

3. The method of claim 2, wherein R₃ and R₄ taken together is OCH₂O.

4. The method of claim 3, wherein R₅ is H.

5. The method of claim 4, wherein Z₁ is CH₂ and Z₂ is C(O).

6. The method of claim 5, wherein R₆ and R₇ taken together is O or R₆ is OH and R₇ is NHCH₃.

7. The method of claim 1, wherein Z₁ is CH₂ and Z₂ is C(O).

8. The method of claim 7, wherein R₆ and R₇ taken together is O or R₆ is OH and R₇ is NHCH₃.

9. The method of claim 8, wherein Ar is phenyl, and is substituted with R₃, R₄, and R₅ at positions 3, 4, and 5, respectively.

10. The method of claim 9, wherein R₁ and R₂ taken together is CH₂.

11. The method of claim 10, wherein R₃ and R₄ taken together is OCH₂O.

12. The method of claim 1, wherein R₁ and R₂ taken together is CH₂.

13. The method of claim 12, wherein Ar is phenyl, and is substituted with R₃, R₄, and R₅ at positions 3, 4, and 5, respectively.

14. The method of claim 13, wherein R₃ and R₄ taken together is OCH₂O.

15. The method of claim 14, wherein R₅ is H.

16. The method of claim 15, wherein Z₁ is CH₂.

17. The method of claim 16, wherein Z₂ is C(O).

18. The method of claim 17, wherein R₆ and R₇ taken together is O or R₆ is OH and R₇ is NHCH₃.

19. The method of claim 16, wherein Z₂ is CH₂.

20. The method of claim 15, wherein Z₁ is C(O).

21. The method of claim 20, wherein Z₂ is C(O).

22. The method of claim 20, wherein Z₂ is CH₂.

23. The method of claim 1, wherein the subject is concurrently administered with an effective amount of a second antiviral compound.

24. The method of claim 23, wherein the second antiviral compound is acyclovir, gancicolovir, famciclovir, cidofovir, foscarnet sodium, penciclovir, valaciclovir, vidarabine, or fomivirsen.

25. The method of claim 1, wherein the β-herpesvirinae is herpesvirus 5.

26. The method of claim 1, wherein the β-herpesvirinae is herpesvirus 6 or 7.

27. A method for treating infection by a γ-herpesvirinae, comprising administering to a subject in need thereof an effective amount of an antiviral compound of formula (I): 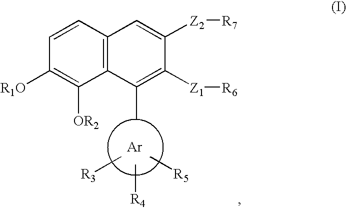
wherein
each of R₁ and R₂, independently is R or C(O)R; or R₁ and R₂ taken together is (CH₂)ₘ;
each of R₃, R₄, and R₅, independently, is R, OR, C(O)R, or OC(O)R; or any two of R₃, R₄, and R₅ taken together is O(CH₂)ₙO;
Ar is aryl;
each of Z₁ and Z₂, independently, is CH₂ or C(O); and
each of R₆ and R₇, independently, is R, OR, SR, or NRR′; or R₆ and R₇ taken together is O, S, or NR;
in which each of R and R′, independently, is H, alkyl, (CH₂)ₒ-aryl, (CH₂)ₚ-heteroaryl, cyclyl, or heterocyclyl; each of m and n, independently, is 1, 2, 3, or 4; and each of o and p, independently, is 0, 1, 2, 3, 4, 5, or 6.

28. The method of claim 27, wherein Ar is phenyl, and is substituted with R₃, R₄, and R₅ at positions 3, 4, and 5, respectively.

29. The method of claim 28, wherein R₃ and R₄ taken together is OCH₂O.

30. The method of claim 29, wherein R₅ is H.

31. The method of claim 30, wherein Z₁ is CH₂ and Z₂ is C(O).

32. The method of claim 31, wherein R₆ and R₇ taken together is O or R₆ is OH and R₇ is NHCH₃.

33. The method of claim 27, wherein Z₁ is CH₂ and Z₂ is C(O).

34. The method of claim 33, wherein R₆ and R₇ taken together is O or R₆ is OH and R₇ is NHCH₃.

35. The method of claim 34, wherein Ar is phenyl, and is substituted with R₃, R₄, and R₅ at positions 3, 4, and 5, respectively.

36. The method of claim 35, wherein R₁ and R₂ taken together is CH₂.

37. The method of claim 36, wherein R₃ and R₄ taken together is OCH₂O.

38. The method of claim 27, wherein R₁ and R₂ taken together is CH₂.

39. The method of claim 38, wherein Ar is phenyl, and is substituted with R₃, R₄, and R₅ at positions 3, 4, and 5, respectively.

40. The method of claim 39, wherein R₃ and R₄ taken together is OCH₂O.

41. The method of claim 40, wherein R₅ is H.

42. The method of claim 41, wherein Z₁ is CH₂.

43. The method of claim 42, wherein Z₂ is C(O).

44. The method of claim 43, wherein R₆ and R₇ taken together is O or R₆ is OH and R₇ is NHCH₃.

45. The method of claim 42, wherein Z₂ is CH₂.

46. The method of claim 41, wherein Z₁ is C(O).

47. The method of claim 46, wherein Z₂ is C(O).

48. The method of claim 46, wherein Z₂ is CH₂.

49. The method of claim 27, wherein the subject is concurrently administered with an effective amount of a second antiviral compound.

50. The method of claim 49, wherein the second antiviral compound is acyclovir, gancicolovir, famciclovir, cidofovir, foscarnet sodium, penciclovir, valaciclovir, vidarabine, or fomivirsen.

51. The method of claim 27, wherein the γ-herpesvirinae is herpesvirus 4.

52. The method of claim 27, wherein the γ-herpesvirinae is herpesvirus 8.

53. A method for treating infection by herpesvirus 3, comprising administering to a subject in need thereof an effective amount of an antiviral compound of formula (I): 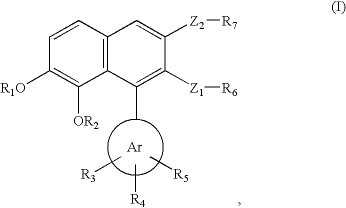
wherein
each of R₁ and R₂, independently is R or C(O)R; or R₁ and R₂ taken together is (CH₂)ₘ;
each of R₃, R₄, and R₅, independently, is R, OR, C(O)R, or OC(O)R; or any two of R₃, R₄, and R₅ taken together is O(CH₂)ₙO;
Ar is aryl;
each of Z₁ and Z₂, independently, is CH₂ or C(O); and
each of R₆ and R₇, independently, is R, OR, SR, or NRR′; or R₆ and R₇ taken together is O, S, or NR;
in which each of R and R′, independently, is H, alkyl, (CH₂)ₒ-aryl, (CH₂)ₚ-heteroaryl, cyclyl, or heterocyclyl; each of m and n, independently, is 1, 2, 3, or 4; and each of o and p, independently, is 0, 1, 2, 3, 4, 5, or 6.

54. The method of claim 53, wherein Ar is phenyl, and is substituted with R₃, R₄, and R₅ at positions 3, 4, and 5, respectively.

55. The method of claim 54, wherein R₃ and R₄ taken together is OCH₂O.

56. The method of claim 55, wherein R₅ is H.

57. The method of claim 56, wherein Z₁ is CH₂ and Z₂ is C(O).

58. The method of claim 57, wherein R₆ and R₇ taken together is 0 or R₆ is OH and R₇ is NHCH₃.

59. The method of claim 53, wherein Z₁ is CH₂ and Z₂ is C(O).

60. The method of claim 59, wherein R₆ and R₇ taken together is O or R₆ is OH and R₇ is NHCH₃.

61. The method of claim 60, wherein Ar is phenyl, and is substituted with R₃, R₄, and R₅ at positions 3, 4, and 5, respectively.

62. The method of claim 61, wherein R₁ and R₂ taken together is CH₂.

63. The method of claim 62, wherein R₃ and R₄ taken together is OCH₂O.

64. The method of claim 53, wherein R₁ and R₂ taken together is CH₂.

65. The method of claim 64, wherein Ar is phenyl, and is substituted with R₃, R₄, and R₅ at positions 3, 4, and 5, respectively.

66. The method of claim 65, wherein R₃ and R₄ taken together is OCH₂O.

67. The method of claim 66, wherein R₅ is H.

68. The method of claim 67, wherein Z₁ is CH₂.

69. The method of claim 68, wherein Z₂ is C(O).

70. The method of claim 69, wherein R₆ and R₇ taken together is O or R₆ is OH and R₇ is NHCH₃.

71. The method of claim 68, wherein Z₂ is CH₂.

72. The method of claim 67, wherein Z₁ is C(O).

73. The method of claim 72, wherein Z₂ is C(O).

74. The method of claim 72, wherein Z₂ is CH₂.

75. A method for treating infection by an α-herpesvirinae, comprising administering to a subject in need thereof an effective amount of a first antiviral compound and an effective amount of a second antiviral compound of formula (I): 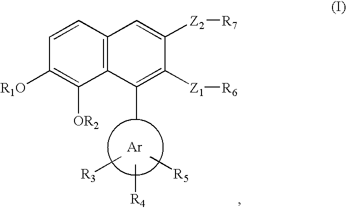
wherein
each of R₁ and R₂, independently is R or C(O)R; or R₁ and R₂ taken together is (CH₂)ₘ;
each of R₃, R₄, and R₅, independently, is R, OR, C(O)R, or OC(O)R; or any two of R₃, R₄, and R₅ taken together is O(CH₂)ₙO;
Ar is aryl;
each of Z₁ and Z₂, independently, is CH₂ or C(O); and
each of R₆ and R₇, independently, is R, OR, SR, or NRR′; or R₆ and R₇ taken together is O, S, or NR;
in which each of R and R′, independently, is H, alkyl, (CH₂)ₒ-aryl, (CH₂)ₚ-heteroaryl, cyclyl, or heterocyclyl; each of m and n, independently, is 1, 2, 3, or 4; and each of o and p, independently, is 0, 1, 2, 3, 4, 5, or 6.

76. The method of claim 75, wherein the first antiviral compound is acyclovir, gancicolovir, famciclovir, cidofovir, foscarnet sodium, penciclovir, valaciclovir, vidarabine, or fomivirsen.

77. The method of claim 75, wherein the α-herpesvirinae is herpesvirus 1.

78. The method of claim 75, wherein the α-herpesvirinae is herpesvirus 2.

79. The method of claim 75, wherein the α-herpesvirinae is herpesvirus 3.
